(19) United States
(12) Patent Application Publication
(10) Pub. No.: US 2018/0162803 A1
(43) Pub. Date: Jun. 14, 2018

(54) METAL COMPLEXES

(71) Applicant: Novus International, Inc., St. Charles, MO (US)

(72) Inventors: John Hume, St. Charles, MO (US); Courtney Moore, St. Charles, MO (US); Sarah Perdue, St. Charles, MO (US)

(21) Appl. No.: 15/839,394

(22) Filed: Dec. 12, 2017

Related U.S. Application Data
(60) Provisional application No. 62/433,058, filed on Dec. 12, 2016.

(51) Int. Cl. C07C 229/76; C07C 227/16; A23K 20/142

(52) U.S. Cl. CPC C07C 229/76; A23K 20/142; C07C 227/16

(57) ABSTRACT

The disclosure provides metal complexes which have improved stability in the presence of humidity. The disclosure also provides a method of making metal complexes and uses thereof.

### CROSS REFERENCE TO RELATED APPLICATION

This application claims priority to U.S. provisional application No. 62/433,058, filed Dec. 12, 2016, which is hereby incorporated by reference in its entirety.

### FIELD OF THE INVENTION

The disclosure provides metal complexes which have improved stability in the presence of humidity. The disclosure also provides a method of making metal complexes and uses thereof.

### SUMMARY OF THE INVENTION

One aspect of the present disclosure is a zinc glycinate complex, the zinc glycinate complex comprising: a zinc cation; an anion derived from an organic or inorganic acid; glycine, wherein the glycine is coordinated with the zinc cation; and one or more water molecules coordinated with the zinc cation; wherein the zinc glycinate complex increases in weight by less than about 3% following exposure to about 60% humidity at room temperature for seven days.

A copper glycinate complex, the copper glycinate complex comprising: a copper cation; an anion derived from an organic or inorganic acid; glycine, wherein the glycine is coordinated with the copper cation; and one or more water molecules coordinated with the copper cation; wherein the copper glycinate complex increases in weight by less than about 2% following exposure to about 60% humidity at room temperature for seven days.

A metal glycinate complex, the metal glycinate complex comprising: a metal cation; an anion derived from an organic or inorganic acid; glycine, wherein the glycine is coordinated with the metal cation; and one or more water molecules coordinated to the metal cation; wherein the metal glycinate complex increases in weight by less than about 3% following humidity exposure.

A feed composition, the feed composition comprising any of the above metal complexes.

A process of making a metal glycinate complex, the process comprising the steps of: mixing the following components: (i) a metal cation and an anion derived from an organic or inorganic acid, (ii) glycine and (iii) a limited amount of water, to generate a metal glycinate complex; heating, while mixing in step (a), to not more than 80° C.; and drying the metal glycinate complex to remove residual water; wherein the product comprising metal glycinate complex increases in weight by less than 3% following humidity exposure at room temperature for seven days.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts a process flow diagram for a continuous metal glycinate complex process. The process is typical for Zn, Mn, Cu and Fe.

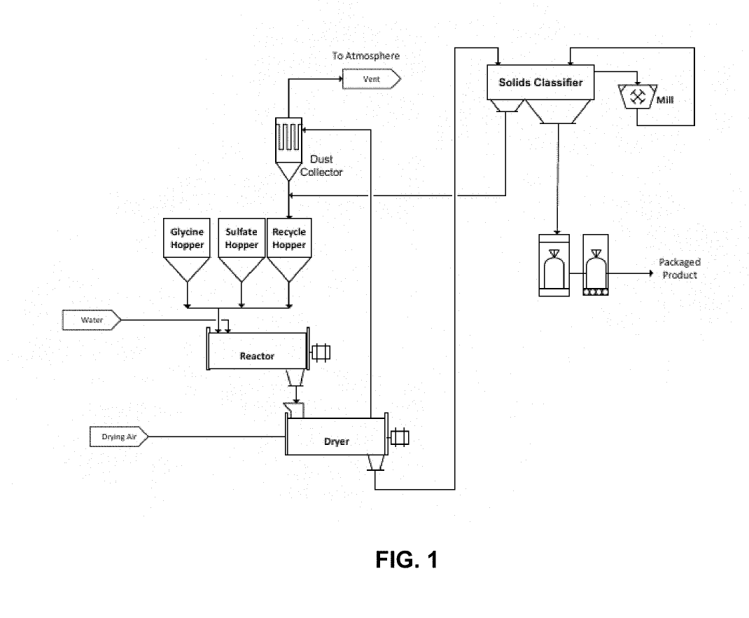

FIG. 2 depicts the crystal packing and hydrogen bonding of a metal glycinate complex of the disclosure. Color codes: red—oxygen, white—hydrogen, light gray—carbon, light purple—nitrogen, yellow—sulfur, and dark gray—metal ion. The cyan dotted lines indicate hydrogen bonds.

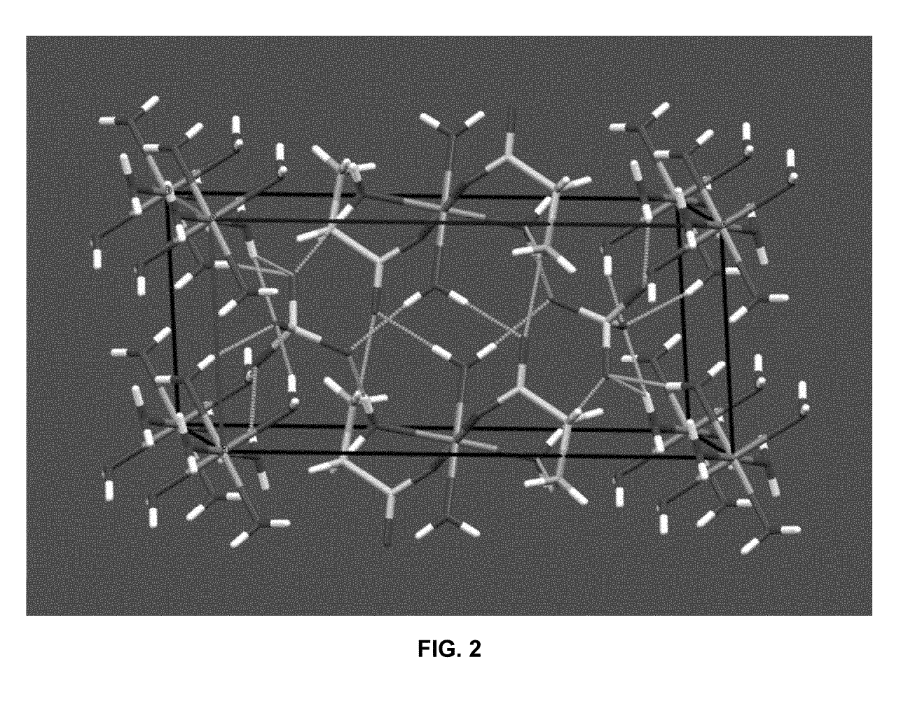

FIG. 3 depicts the results of a thermogravimetric analysis (TGA) comparison of zinc glycinate complexes of this disclosure (identified as “prototypes”), competitor zinc glycinate complexes, raw materials (ZnSO₄—H₂O, glycine, and 1:1 molar mixture of glycine to ZnSO₄—H₂O. (High resolution 4, 10° C./min ramp to 450° C., N₂ atmosphere).

### DETAILED DESCRIPTION OF THE INVENTION

The disclosure provides metal complexes, processes of making metal complexes, and uses of metal complexes. The metal complexes disclosed herein have increased stability due to reduced free water in the final product. The inventors have discovered the appropriate hydration of the metal complexes to reduce moisture gain and hygroscopicity. Moisture gain and hygroscopicity are undesirable as this could change the product composition and lead to reduced shelf-life (caking issues). The process of making metal complexes described herein allows for the selection of the final product hydration value which is important to reducing the hygroscopicity of the metal complex. The process also does not produce an aqueous waste stream, thereby eliminating the need to filter, evaporate or otherwise dispose of an aqueous waste stream. As such, the process is more sustainable or environmentally friendly than methods known in the art.

### I. Metal Complex

In an aspect, the disclosure provides a metal complex. The metal complex comprises: a metal cation, an anion derived from an organic or inorganic acid; and an amino acid, wherein the amino acid is coordinated with the metal cation; and wherein the metal complex increases in weight by less than 5% following humidity exposure. In another aspect, the metal complex comprises: a metal cation, an anion derived from an organic or inorganic acid; and glycine, wherein the glycine is coordinated with the metal cation; and wherein the metal complex increases in weight by less than 3% following humidity exposure.

As used herein, a metal cation is a positively charged ion that forms when a metal loses electrons. A metal cation has the general formula Me⁺ⁿ, wherein Me is a metal and +n is a number from 1 to 10. Any suitable metal cation may be included in a metal glycinate complex. Non-limiting examples of metals that may be included in a metal glycinate complex include calcium, magnesium, zinc, iron, copper, manganese, sodium, potassium, cobalt, nickel, chromium, molybdenum, germanium, lithium, rubidium, tin, and vanadium. Specifically, the metal cation may be selected from the group consisting of calcium, magnesium, zinc, iron, copper, manganese, sodium, potassium, cobalt, and nickel. More specifically, the metal cation may be selected from the group consisting of zinc, iron, copper, and manganese.

As used herein, an anion derived from an organic or inorganic acid is a negatively charged ion that forms when an organic acid or inorganic acid loses a proton. An anion has the general formula A⁻ⁿ, wherein A is an anion and −n is a number from 1 to 10. As used herein, an organic acid is an organic compound with acidic properties. The most common organic acids are carboxylic acid whose acidity is associated with a carboxyl group (—COOH). Non-limiting examples of organic acids include formic acid (methanoic acid), acetic acid (ethanoic acid), propionic acid (propanoic acid), butyric acid (butanoic acid), valeric acid (pentanoic acid), caproic acid (hexanoic acid), oxalic acid (ethanedioic acid), lactic acid (2-hydroxypropanoic acid), malic acid (2-hydroxybutanedioic acid), fumaric acid (trans-butenedioic acid), citric acid (2-hydroxypropane-1,2,3-tricarboxylic acid), gluconic acid, benzoic acid (benzenecarboxylic acid or phenylmethanoic acid), 2-hydroxy-4-(methythio)butyric acid, and carbonic acid (hydroxymethanoic acid). As used herein, an inorganic acid, also referred to as a mineral acid, is an acid derived from one or more inorganic compounds. An inorganic acid forms hydrogen ions and the conjugate base ions when dissolved in water. Non-limiting examples of inorganic acids include sulfuric acid (H₂SO₄), hydrochloric acid (HCl), nitric acid (HNO₃), boric acid (H₃BO₃), phosphoric acid (H₃PO₄), hydrofluoric acid (HF), hydrobromic acid (HBr), perchloric acid (HClO₄), and hydroioidic acid (Hl). Specifically, an anion is derived from an organic or inorganic acid selected from the group consisting of formic acid, acetic acid, fumaric acid, malic acid, hydrochloric acid, phosphoric acid, and sulfuric acid. More specifically, an anion is derived from sulfuric acid. When the anion is derived from sulfuric acid, it is referred to as a sulfate (SO₄⁻²) anion.

As used herein glycine is an amino acid having the formula NH₂CH₂COOH. Glycine is the smallest of the 20 amino acids commonly found in proteins. In the embodiments described herein, glycine is coordinated with the metal cation to form a metal glycinate complex. When a metal salt (e.g. metal cation and anion derived from an organic or inorganic acid) is contacted with glycine under the proper conditions, a complex is formed. The lattice formed between the glycine, metal cation, the corresponding anion, and waters of hydration results in a crystalline structure that is readily soluble in water. Other amino acids may also be used to form a metal complex. Non-limiting examples of amino acids include arginine, histidine, lysine, aspartic acid, glutamic acid, serine, threonine, asparagine, glutamine, cysteine, selenocysteine, proline, alanine, isoleucine, leucine, methionine, selenomethionine, phenylalanine, tryptophan, tyrosine, and valine.

A metal complex may further comprise water coordinated with the metal cation. Accordingly, one or more water molecules may be bound to the metal cation or other species in the complex. The compound comprising a metal cation, an anion, and amino acid, and one or more water molecules is referred to as a hydrate. A hydrate has the general formula Me⁺ⁿGlyA⁻ⁿ.xH₂O, wherein Me is a metal cation, Gly is glycine or any other amino acid, A is an anion, n is an integer from about 1 to about 10, and x is an integer from about 1 to about 12. For example, a hydrate may be a monohydrate (x=1), a dihydrate (x=2), a trihydrate (x=3), a tetrahydate (x=4), a pentahydrate (x=5), a hexahydrate (x=6), a heptahydrate (x=7), an octahydrate (x=8), a nonahydrate (x=9), a decahydrate (x=10), an undecahydrate (x=11), and a dodecahydrate (x=12). Occasionally, a hydrate may be fractional such as a hemihydrate (x=1/2) or a sequihydrate (x=1 ¹/₂). In an aspect, a metal complex may comprise about 1 to about 5 moles of water per mole of metal complex. In another aspect, a metal complex may comprise 1 to 3 moles of water per mole of metal complex. In still another aspect, a metal glycinate complex may comprise about 1 to about 5 moles of water per mole of metal glycinate complex. In a specific aspect, a metal glycinate complex may comprise 1 to 3 moles of water per mole of metal glycinate complex.

Exemplary metal glycinate complexes include zinc glycinate trihydrate [Zn(NH₂CH₂COOH)(SO₄).3H₂O], zinc glycinate pentahydrate [Zn(NH₂CH₂COOH)(SO₄).5H₂O], manganese glycinate [Mn(NH₂CH₂COOH)(SO₄)], copper glycinate dihydrate [Cu(NH₂CH₂COOH)(SO₄).2H₂O], ferrous glycinate trihydrate [Fe(NH₂CH₂COOH)(SO₄).3H₂O], and ferrous glycinate pentahydrate [Fe(NH₂CH₂COOH)(SO₄).5H₂O]. The aforementioned metal glycinate hydrates have the appropriate hydration to reduce moisture gain and hygroscopicity.

Importantly, the metal complex of the disclosure has increased stability in the presence of humidity. As used herein, “humidity” may refer to water in the atmosphere. The stability may be measured by the increase in weight of the metal complex following humidity exposure. A metal complex increases in weight by less than about 5% following humidity exposure. More specifically, a metal complex increases in weight by less than about 5% following exposure to 60% relative humidity at 25° C. for about 1 day to about 7 days. For example, a metal complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 60% relative humidity at 25° C. for about 1 day to about 7 days. Additionally, a metal complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 60% relative humidity at 25° C. for about 7 days. In a specific embodiment, a metal complex increases in weight by less than about 2% following exposure to 60% relative humidity at 25° C. for about 7 days.

In another aspect, a metal complex increases in weight by less than about 5% following exposure to 75% relative humidity at 45° C. for about 1 day to about 7 days. For example, a metal complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 75% relative humidity at 45° C. for about 1 day to about 7 days. Additionally, a metal complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 75% relative humidity at 45° C. for about 7 days. In a specific embodiment, a metal complex increases in weight by less than about 3% following exposure to 75% relative humidity at 45° C. for about 7 days.

In still another aspect, a metal glycinate complex increases in weight by less than about 5% following humidity exposure. More specifically, a metal glycinate complex increases in weight by less than about 5% following exposure to 60% relative humidity at 25° C. for about 1 day to about 7 days. For example, a metal glycinate complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 60% relative humidity at 25° C. for about 1 day to about 7 days. Additionally, a metal glycinate complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 60% relative humidity at 25° C. for about 7 days. In a specific embodiment, a metal glycinate complex increases in weight by less than about 2% following exposure to 60% relative humidity at 25° C. for about 7 days.

In another aspect, a metal glycinate complex increases in weight by less than about 5% following exposure to 75% relative humidity at 45° C. for about 1 day to about 7 days. For example, a metal glycinate complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 75% relative humidity at 45° C. for about 1 day to about 7 days. Additionally, a metal complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 75% relative humidity at 45° C. for about 7 days. In a specific embodiment, a metal glycinate complex increases in weight by less than about 3% following exposure to 75% relative humidity at 45° C. for about 7 days.

In certain aspects, zinc glycinate trihydrate or zinc glycinate pentahydrate increases in weight by less than 3%, less than 2.5%, less than 2%, less than 1.5%, less than 1.4%, less than 1.3%, less than 1.2%, less than 1.1%, less than 1%, less than 0.9%, less than 0.8%, less than 0.7%, less than 0.6%, or less than 0.5% following exposure to 60% relative humidity at 25° C. for about 7 days. Additionally, zinc glycinate trihydrate or zinc glycinate pentahydrate increases in weight by less than 5%, less than 4.5%, less than 4%, less than 3.5%, less than 3%, less than 2.5%, or less than 2% following exposure to 75% relative humidity at 45° C. for about 7 days.

In other aspects, manganese glycinate increases in weight by less than 1%, less than 0.9%, less than 0.8%, less than 0.7%, less than 0.6%, less than 0.5%, less than 0.4%, less than 0.3%, less than 0.2%, or less than 0.1% following exposure to 60% relative humidity at 25° C. for about 7 days. Additionally, manganese glycinate increases in weight by less than 1%, less than 0.9%, less than 0.8%, less than 0.7%, less than 0.6%, less than 0.5%, less than 0.4%, less than 0.3%, less than 0.2%, or less than 0.1% following exposure to 75% relative humidity at 45° C. for about 7 days.

In still other aspects, copper glycinate dihydrate increases in weight by less than 3%, less than 2.5%, less than 2%, less than 1.5%, less than 1%, less than 0.9%, less than 0.8%, less than 0.7%, less than 0.6%, less than 0.5%, less than 0.4%, less than 0.3%, less than 0.2%, or less than 0.1% following exposure to 60% relative humidity at 25° C. for about 7 days. Additionally, copper glycinate dihydrate increases in weight by less than 5%, less than 4.5%, less than 4%, less than 3.5%, less than 3%, less than 2.5%, less than 2%, less than 1.5%, less than 1%, less than 0.9%, less than 0.8%, less than 0.7%, less than 0.6%, less than 0.5%, less than 0.4%, less than 0.3%, less than 0.2%, or less than 0.1% following exposure to 75% relative humidity at 45° C. for about 7 days.

In still yet other aspects, ferrous glycinate trihydrate or ferrous glycinate pentahydrate increases in weight by less than 1.5%, less than 1%, less than 0.9%, less than 0.8%, less than 0.7%, less than 0.6%, less than 0.5%, less than 0.4%, less than 0.3%, less than 0.2%, or less than 0.1% following exposure to 60% relative humidity at 25° C. for about 7 days. Additionally, ferrous glycinate trihydrate or ferrous glycinate pentahydrate increases in weight by less than 2.5%, less than 2%, less than 1.5%, less than 1%, less than 0.9%, less than 0.8%, less than 0.7%, less than 0.6%, less than 0.5%, less than 0.4%, less than 0.3%, less than 0.2%, or less than 0.1% following exposure to 75% relative humidity at 45° C. for about 7 days.

### II. Feed Compositions

One aspect of the present disclosure encompasses a feed composition comprising a metal complex described herein. Suitable feed compositions may be formulated for agriculture animals, companion animals, or non-domesticated animals.

### III. Process of Making a Metal Complex

In an aspect, the disclosure provides a process of making a metal complex. The process comprises the steps of: (a) mixing (i) a metal cation and an anion derived from an organic or inorganic acid, (ii) an amino acid and (iii) a limited amount of water in a mixer to generate a metal complex; and (b) drying the metal complex to remove residual water, wherein the product comprising metal complex increases in weight by less than 5% following humidity exposure. The metal complex may be further milled to obtain a product comprising metal complex of a desired particle size. In another aspect, the disclosure provides a process of making a metal glycinate complex. The process comprises the steps of: (a) mixing (i) a metal cation and an anion derived from an organic or inorganic acid, (ii) glycine and (iii) a limited amount of water in a mixer to generate a metal glycinate complex; and (b) drying the metal glycinate complex to remove residual water, wherein the product comprising metal glycinate complex increases in weight by less than 3% following humidity exposure. The metal glycinate complex may be milled to obtain a product comprising metal glycinate complex of a desired particle size. In the foregoing processes of making, the mixer may be open to the atmosphere. The aforementioned processes allow for the selection of the hydration value of the final metal complex. The selection of appropriate hydration value of the final metal complex reduces the hygroscopicity of the metal complex.

Amino acids/glycine and the metal cation and anion derived from an organic or inorganic acid are as described in Section I. The metal cation and anion derived from an organic or inorganic acid may further comprise water coordinated with the metal cation. Accordingly, one or more water molecules may be bound to the metal cation. The compound comprising a metal cation, an anion, and one or more water molecules is referred to as a hydrate. A hydrate has the general formula Me⁺ⁿA⁺ⁿ.xH₂O, wherein Me is a metal cation, A is an anion, n is an integer from about 1 to about 10, and x is an integer from about 1 to about 12. For example, a hydrate may be a monohydrate (x=1), a dihydrate (x=2), a trihydrate (x=3), a tetrahydate (x=4), a pentahydrate (x=5), a hexahydrate (x=6), a heptahydrate (x=7), an octahydrate (x=8), a nonahydrate (x=9), a decahydrate (x=10), an undecahydrate (x=11), and a dodecahydrate (x=12). Occasionally, a hydrate may be fractional such as a hemihydrate (x=½) or a sequihydrate (x=1½). Specifically, a metal hydrate may comprise about 1 to about 10 moles of water per mole of metal cation. More specifically, a metal hydrate may comprise 1 to 7 moles of water per mole of metal cation. Exemplary metal cation and anion derived from an organic or inorganic acid compounds include zinc sulfate monohydrate [ZnSO₄.H₂O], manganese sulfate monohydrate [MnSO₄.H₂O], copper sulfate pentahydrate [CuSO₄.5H₂O], and ferrous sulfate heptahydrate [FeSO₄/7H₂O].

The metal cation and an anion derived from an organic or inorganic acid, amino acid and water are mixed in a mixer to generate a metal complex. Specifically, the metal cation and an anion derived from an organic or inorganic acid, glycine and water are mixed in a mixer to generate a metal glycinate complex. In each of the foregoing embodiments, the mixer may be open to the atmosphere. The metal cation and amino acid are mixed in about a 1:1 molar ratio. For example, the metal cation and amino acid may be mixed in about a 2:1, about a 1.9:1, about a 1.8:1, about a 1.7:1, about a 1.6:1, about a 1.5:1, about a 1.4:1, about a 1.3:1, about a 1.2:1, about a 1.1:1, about a 1:1.1, about a 1:1.2, about a 1:1.3, about a 1:1.4, about a 1:1.5, about a 1:1.6, about a 1:1.7, about a 1:1.8, about a 1:1.9, about a 1:2, about a 1:3, about a 1:4, or about a 1:5 molar ratio. In certain embodiments, the metal cation and glycine are mixed in about a 1:1 molar ratio. For example, the metal cation and glycine may be mixed in about a 2:1, about a 1.9:1, about a 1.8:1, about a 1.7:1, about a 1.6:1, about a 1.5:1, about a 1.4:1, about a 1.3:1, about a 1.2:1, about a 1.1:1, about a 1:1.1, about a 1:1.2, about a 1:1.3, about a 1:1.4, about a 1:1.5, about a 1:1.6, about a 1:1.7, about a 1:1.8, about a 1:1.9, about a 1:2, about a 1:3, about a 1:4, or about a 1:5 molar ratio.

As the metal cation and anion derived from an organic or inorganic acid and amino acid are mixed, a limited amount of water is added to the mixture to generate a metal complex. Specifically, as the metal cation and anion derived from an organic or inorganic acid and glycine are mixed, a limited amount of water is added to the mixture to generate a metal glycinate complex. The water may be sprayed onto the solids to achieve flowability of the mixture. As used herein, a “limited amount” of water refers to about 0.1 wt % to about 35 wt % water relative to the metal cation and anion derived from an organic or inorganic acid and amino acid/glycine. The limited amount of water excludes potential water available from the metal hydrates. The amount of water added is designed to facilitate formation of the desired metal complex hydrate. In one embodiment, less than about 5, 10, 15, 20, 25, 30, or 35 wt % water is used. In another embodiment, about 0.1 wt % to about 10wt % water is used. In yet another embodiment, about 10 wt % to about 20 wt % water is used. In still another embodiment, about 20 wt % to about 30 wt % water is used. In another embodiment, about 30 wt % to about 35 wt % water is used. In some embodiments, about 0.1 to about 1 wt %, about 5 to about 10 wt %, about 10 to about 15 wt %, and about 20 to about 25 wt % water is used.

Mixing occurs at a reaction temperature of about 25° C. to about 80° C. To achieve the desired reaction temperature, external heat may or may not be added during mixing. If the heat of solution is exothermic, it may be sufficient to drive the reaction without applying external heat. Alternatively, if the heat of solution is not exothermic or exothermic but not sufficient to drive the reaction, then external heat may need to be applied. External heat should be applied to achieve a reaction temperature of about 25° C. to about 80° C. In an exemplary embodiment, when the metal cation and anion derived from an organic or inorganic acid is zinc sulfate monohydrate or copper sulfate pentahydrate, no external heat is added during mixing. In another exemplary embodiment, when the metal cation and anion derived from an organic or inorganic acid is manganese sulfate monohydrate or ferrous sulfate heptahydate, external heat is added during mixing. In some embodiment, the reaction temperature is less than 60° C., less than 55° C., less than 50° C., or less than 45° C.

Mixing occurs for at least 30 minutes. Mixing may occur for about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, or about 120 minutes. In alternative embodiments, mixing may occur for longer than 120 minutes.

The metal complex is then dried to remove residual water. Drying may occur by any suitable method, or combination of methods, known in the art. For example, drying may occur by drying in an open batch or continuous agitated mixer under direct heat; drying in an open open batch or continuous agitated mixer under indirect heat; drying in an open open batch or continuous agitated mixer under vacuum; or fluid bed drying. The metal complex may be dried at less than 200° C. Alternatively, the metal complex may be dried at less than 110° C. Further, the metal complex may be dried at less than 70° C. Still further, the metal complex may be dried at less than 50° C. The specific drying temperature may be chosen based on the metal complex to be dried. For example, the metal complex may be dried at less than 200° C., less than 190° C., less than 180° C., less than 170° C., less than 160° C., less than 150° C., less than 140° C., less than 130° C., less than 120° C., less than 110° C., less than 100° C., less than 90° C., less than 80° C., less than 70° C., less than 60° C., less than 50° C., less than 40° C., less than 30° C., or less than 20° C. In exemplary embodiments, a suitable drying temperature for the metal complex is a temperature that removes any free moisture without breaking the particular hydrate formed. In an exemplary embodiment, when the metal glycinate complex comprises zinc, the metal glycinate complex may be dried at less than 50° C. In another exemplary embodiment, when the metal glycinate complex comprises manganese, the metal glycinate complex may be dried at less than 200° C. In still another exemplary embodiment, when the metal glycinate complex comprises copper, the metal glycinate complex may be dried at less than 110° C. In still yet another exemplary embodiment, when the metal glycinate complex comprises iron, the metal glycinate complex may be dried at less than 70° C.

In certain embodiments when the metal cation is zinc, manganese, copper or iron, the limited amount of water added to the mixture, the reaction temperature and the drying temperature may be as follows. In these embodiments, “about” refers to ±2-5%.

| | | | | Total moisture |
|---|---|---|---|---|
| | Wt % | Reaction | Drying | in the metal |
| Metal | water added | temp. | temp. | glycinate |
| Zn | about 23% | ≤60° C. | ≤50° C. | about 18% |
| Mn | about 11% | ≤80° C. | ≤200° C. | about 2% |
| Cu | about 0.5% | ≤47° C. | ≤70° C. | about 14% |
| Fe | about 6.6% | ≤55° C. | ≤70° C. | about 20% |

In certain embodiments when the metal cation is zinc, manganese, copper or iron, the limited amount of water added to the mixture, the reaction temperature and the drying temperature may be as follows. In these embodiments, “about” refers to ±2-5%.

| | | | | Total moisture |
|---|---|---|---|---|
| | Wt % | Reaction | Drying | in the metal |
| Metal | water added | temp. | temp. | glycinate |
| Zn | about 23% | about 35° C. to | ≤50° C. | about 18% |
| | | about 50° C. | | |
| Mn | about 11% | about 60° C. to | ≤200° C. | about 2% |
| | | about 75° C. | | |
| Cu | about 0.5% | about 37° C. to | ≤70° C. | about 14% |
| | | about 47° C. | | |
| Fe | about 6.6% | about 42-52° C. | ≤70° C. | about 20% |

In certain embodiments when the metal cation is zinc, manganese, copper or iron, the limited amount of water added to the mixture, the reaction temperature and the drying temperature may be as follows. In these embodiments, “about” refers to ±2-5%.

| | | | | Total moisture |
|---|---|---|---|---|
| | Wt % | Reaction | Drying | in the metal |
| Metal | water added | temp. | temp. | glycinate |
| Zn | about 23% | about 35° C. to | about 30° C. to | about 18% |
| | | about 50° C. | about 45° C. | |
| Mn | about 11% | about 60° C. to | about 25° C. to | about 2% |
| | | about 75° C. | about 200° C. | |
| Cu | about 0.5% | about 37° C. to | about 25° C. to | about 14% |
| | | about 47° C. | about 70° C. | |
| Fe | about 6.6% | about 42-52° C. | about 50° C. to | about 20% |
| | | | about 70° C. | |

The metal complex may be milled to obtain a product comprising metal complex of a desired particle size. Methods of milling are standard in the art. For example, a vibrating screen system may be used. A screening system comprises at least two screens: a coarse screen and a fine screen. The coarse screen separates the oversized material from the product and the fine screen removes small particle size material that would make the product dusty. The coarse screen may be 600 micron, 30 US MESH. The fine screen may be 104 micron, 140 US MESH. Accordingly, a particles size of metal complex may range from about 104 microns to about 600 microns. A skilled artisan is able to choose a desired particle size based on the downstream use of the product.

Importantly, the product comprising the metal complex increases in weight by less than 5% following humidity exposure at room temperature for seven days. As used herein, “room temperature” refers to about 25° C., for instance between about 20° C. to about 30° C., or about 20, 21, 22, 23, 24, 25, 26, 27, 28, 29, or 30° C. Additionally, the product comprising the metal glycinate complex increases in weight by less than 3% following humidity exposure at room temperature for seven days. The improved stability of the metal complex afforded by the process disclosed herein prevents caking and flow problems of the product. Additionally, the process reduces shipping costs and increases metal content. More specifically, a metal complex increases in weight by less than about 5% following exposure to 60% relative humidity at 25° C. for about 1 day to about 7 days. For example, a metal complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 60% relative humidity at 25° C. for about 1 day to about 7 days. Additionally, a metal complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 60% relative humidity at 25° C. for about 7 days. In a specific embodiment, a metal complex increases in weight by less than about 2% following exposure to 60% relative humidity at 25° C. for about 7 days.

In another aspect, a metal complex increases in weight by less than about 5% following exposure to 75% relative humidity at 45° C. for about 1 day to about 7 days. For example, a metal complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 75% relative humidity at 45° C. for about 1 day to about 7 days. Additionally, a metal complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 75% relative humidity at 45° C. for about 7 days. In a specific embodiment, a metal complex increases in weight by less than about 3% following exposure to 75% relative humidity at 45° C. for about 7 days.

In still another aspect, a metal glycinate complex increases in weight by less than about 5% following humidity exposure. More specifically, a metal glycinate complex increases in weight by less than about 5% following exposure to 60% relative humidity at 25° C. for about 1 day to about 7 days. For example, a metal glycinate complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 60% relative humidity at 25° C. for about 1 day to about 7 days. Additionally, a metal glycinate complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 60% relative humidity at 25° C. for about 7 days. In a specific embodiment, a metal glycinate complex increases in weight by less than about 2% following exposure to 60% relative humidity at 25° C. for about 7 days.

In another aspect, a metal glycinate complex increases in weight by less than about 5% following exposure to 75% relative humidity at 45° C. for about 1 day to about 7 days. For example, a metal glycinate complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 75% relative humidity at 45° C. for about 1 day to about 7 days. Additionally, a metal complex increases in weight by less than about 5%, about 4.5%, about 4%, about 3.5%, about 3%, about 2.5%, about 2%, about 1.9%, about 1.8%, about 1.7%, about 1.6%, about 1.5%, about 1.4%, about 1.3%, about 1.2%, about 1.1%, about 1%, about 0.9%, about 0.8%, about 0.7%, about 0.6%, about 0.5%, about 0.4%, about 0.3%, about 0.2%, or about 0.1% following exposure to 75% relative humidity at 45° C. for about 7 days. In a specific embodiment, a metal glycinate complex increases in weight by less than about 3% following exposure to 75% relative humidity at 45° C. for about 7 days.

Exemplary metal glycinate complexes may include zinc glycinate trihydrate [Zn(NH₂CH₂COOH)(SO₄).3H₂O], zinc glycinate pentahydrate [Zn(NH₂CH₂COOH)(SO₄).5H₂O], manganese glycinate [Mn(NH₂CH₂COOH)(SO₄)], copper glycinate dihydrate [Cu(NH₂CH₂COOH)(SO₄).2H₂O], ferrous glycinate trihydrate [Fe(NH₂CH₂COOH)(SO₄).3H₂O], and ferrous glycinate pentahydrate [Fe(NH₂CH₂COOH)(SO₄)5H₂O].

In certain aspects, zinc glycinate trihydrate or zinc glycinate pentahydrate increases in weight by less than 3%, less than 2.5%, less than 2%, less than 1.5%, less than 1.4%, less than 1.3%, less than 1.2%, less than 1.1%, less than 1%, less than 0.9%, less than 0.8%, less than 0.7%, less than 0.6%, or less than 0.5% following exposure to 60% relative humidity at 25° C. for about 7 days. Additionally, zinc glycinate trihydrate or zinc glycinate pentahydrate increases in weight by less than 5%, less than 4.5%, less than 4%, less than 3.5%, less than 3%, less than 2.5%, or less than 2% following exposure to 75% relative humidity at 45° C. for about 7 days. In other aspects, manganese glycinate increases in weight by less than 1%, less than 0.9%, less than 0.8%, less than 0.7%, less than 0.6%, less than 0.5%, less than 0.4%, less than 0.3%, less than 0.2%, or less than 0.1% following exposure to 60% relative humidity at 25° C. for about 7 days. Additionally, manganese glycinate increases in weight by less than 1%, less than 0.9%, less than 0.8%, less than 0.7%, less than 0.6%, less than 0.5%, less than 0.4%, less than 0.3%, less than 0.2%, or less than 0.1% following exposure to 75% relative humidity at 45° C. for about 7 days. In still other aspects, copper glycinate dihydrate increases in weight by less than 3%, less than 2.5%, less than 2%, less than 1.5%, less than 1%, less than 0.9%, less than 0.8%, less than 0.7%, less than 0.6%, less than 0.5%, less than 0.4%, less than 0.3%, less than 0.2%, or less than 0.1% following exposure to 60% relative humidity at 25° C. for about 7 days. Additionally, copper glycinate dihydrate increases in weight by less than 5%, less than 4.5%, less than 4%, less than 3.5%, less than 3%, less than 2.5%, less than 2%, less than 1.5%, less than 1%, less than 0.9%, less than 0.8%, less than 0.7%, less than 0.6%, less than 0.5%, less than 0.4%, less than 0.3%, less than 0.2%, or less than 0.1% following exposure to 75% relative humidity at 45° C. for about 7 days. In still yet other aspects, ferrous glycinate trihydrate or ferrous glycinate pentahydrate increases in weight by less than 1.5%, less than 1%, less than 0.9%, less than 0.8%, less than 0.7%, less than 0.6%, less than 0.5%, less than 0.4%, less than 0.3%, less than 0.2%, or less than 0.1% following exposure to 60% relative humidity at 25° C. for about 7 days. Additionally, ferrous glycinate trihydrate or ferrous glycinate pentahydrate increases in weight by less than 2.5%, less than 2%, less than 1.5%, less than 1%, less than 0.9%, less than 0.8%, less than 0.7%, less than 0.6%, less than 0.5%, less than 0.4%, less than 0.3%, less than 0.2%, or less than 0.1% following exposure to 75% relative humidity at 45° C. for about 7 days.

Another advantage of the process of this disclosure is that the process does not produce an aqueous waste stream. In contrast to the methods in the prior art which require the metals to be dissolved in water, in the current process a metal cation and an anion derived from an organic or inorganic acid, an amino acid and a limited water are mixed in a mixer to generate a metal complex. By minimizing water addition in such a way that the majority of the water is bound to the complex during the reaction, the process minimizes the amount of heat input required for the reaction or drying, and eliminates the need to filter, evaporate or otherwise dispose of an aqueous waste stream.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Zinc Glycinate Complex

Zinc sulfate monohydrate, glycine, and water were continuously fed to a Hydramix IR-300 with the following flowrates: 48 kg/hr zinc sulfate monohydrate; 20 kg/hr glycine, and 14.3 kg/hr water. No external heat was provided and the residence time was 28 minutes. The peak temperature in the reactor was 57° C. The material was then continuously flowed into a vibrating fluid bed dryer with an inlet air temperature of 28° C. The resulting product was granular and free flowing with a zinc content of 22.5 wt % and a total moisture of 18 wt % water corresponding to the trihydrate of the zinc glycinate complex.

Moisture gain and hygroscopicity after manufacturing are undesirable as this could change the product composition and lead to reduced shelf-life (caking issues). Hygroscopicity was evaluated by performing moisture gain and caking tests under room temperature (RT: 25° C., 60% relative humidity) and accelerated conditions (AT: 45° C., 75% relative humidity) from one to seven days. By comparing a zinc glycinate complex produced as described in above to commercially available zinc glycinates, it was confirmed that the process disclosed herein produces a zinc glycinate hydrate that is different than the commercially available complexes.

Table 1, for example, is a summary of the room temperature results up to seven days. Competitor Product 1 and Competitor Product 2 gained significant moisture at room temperature. Product 3, prepared by the process disclosed herein, gained low moisture. These results suggest that the Product 3 is more stable, in relation to water absorption, than Competitor Product 1 and Product 2.

TABLE 1
Moisture Gain and Caking Results from Room Temperature
(RT: 25° C., 60% relative humidity) Tests.

| | Avg Weight Gain (wt %)* | | |
|---|---|---|---|
| Sample Description | 1 day | 3 day | 7 day** |
| Competitor Product 1 | 5.9 | 8.3 | 10.6 |
| Competitor Product 2 | 3.8 | 4.5 | 5.1 |
| Product 3 | 1.1 | 0.9 | 0.9 |

*Avg. Weight Gain (wt %) is calculated using day 0 as baseline.
**Gain CV (7 day; wt %) was 2 for Product 1, 1.6 for Product 2, and 13 for Product 3. CV = Coefficient of Variance = Standard Deviation ÷ Average

Table 2 is a summary of the accelerated humidity (AT) results for samples tested. The pre-test moisture estimate demonstrates that the competitor products are different hydrates than the Product 3. (The moisture content estimate for a pure zinc glycinate mono-, di-, tri-, tetra- and penta-hydrate is 7.1, 13.2, 18.6, 23.4 and 27.6%, respectively.) The average weight gain values, and the Pre-Test Moisure +7 day gain values, show that the competitor products gained significant moisture and Product 3 gained significantly less moisture than the others. These results are consistent with RT results and suggest the competitor products are not moisture stable products.

TABLE 2
Moisture Gain and Caking Results from
45° C. and 75% Relative Humidity Test.

| | | | | Pre-Test | Pre-Test |
|---|---|---|---|---|---|
| | | | | Moisture | Moisture + 7 |
| Sample | Avg. Weight Gain (wt %) | | | Estimate | day Gain |
| Description | 1 day | 3 day | 7 day | wt % | wt % |
| Competitor | 28.9 | 30.6 | 29.1 | 4.58 | 33.7 |
| Product 1 | | | | | |
| Competitor | 14.1 | 16.0 | 16.1 | 1.53 | 17.7 |
| Product 2 | | | | | |
| Product 3 | 4.0 | 5.2 | 2.5 | 16.5 | 19.0 |

* Weight Gain (wt %) is calculated using day 0 as baseline.
** Gain CV (7 day; wt %) was 0.1 for Product 1; 1.9 for Product 2; and 14 for Product 3. CV = Coefficient of Variance = Standard Deviation ÷ Average

Thermogravimetric analysis (TGA) and loss on drying (LOD) were used to quantify the amount of water of hydration, to evaluate hydrate/product degradation for three prototypes produced by the process described above and several competitor products, and to further refine the process conditions. TGA/LOD estimates indicate that the prototypes generated by the process have close to three moles of water per mole of product (between 16-18% moisture).

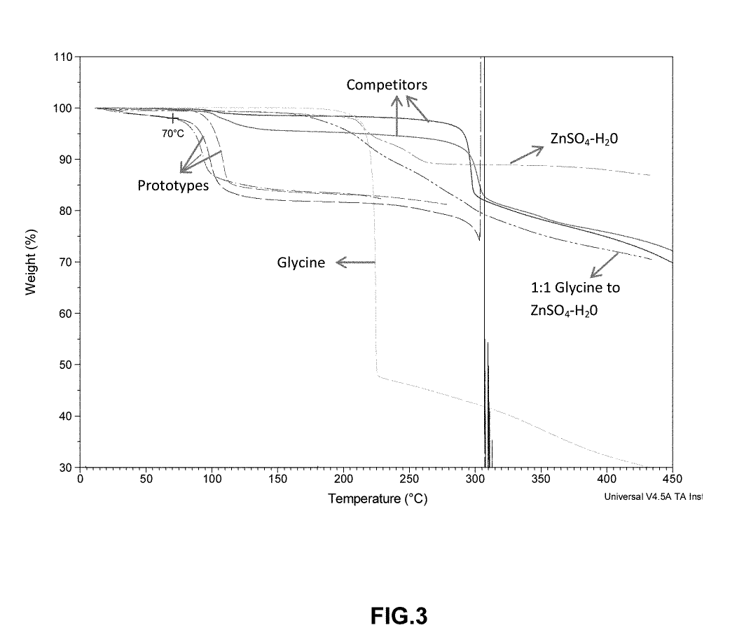

By TGA, the initial weight loss for the prototype and competitor samples between 20-175° C. is where it appears that the majority of the hydrate breaks and where the most weight loss variance is seen between zinc glycinate samples. As shown in FIG. 3, similar onset temperatures for weight loss are observed for the 3 prototypes, suggesting comparable product composition. The onset temperature for the competitor products was different.

By TGA, the onset for rapid hydrate break of the prototypes is approximately 100° C., but slow degradation at lower temperatures appears to occur. The hydrate started breaking at as low as 50° C. but the rate was rather slow at 0.02 wt %/min. The degradation rate increased with increasing temperature. Temperatures of 100° C. and higher have rapid degradation rates (>0.1 wt %/min). Based on this analysis, it was determined that process temperatures, such as reaction temperature and drying temperature, of 100° C. and higher should be avoided. To avoid partial hydrate break, the process utilizes lower temperatures and avoids excess heating/drying. Preferred temperatures are <70° C., preferably <60° C., more preferably about 55° C. or less. Current process development has been able to minimize water addition in such a way to bind the majority of the water to the complex during the reaction and use the heat of solution to provide the drive the reaction; thus, minimizing the amount of heat input required for the reaction or drying

### Example 2

### Manganese Glycinate Complex

Manganese sulfate monohydrate, glycine, and water were continuously fed to a Hydramix IR-300 with the following flowrates: 200 lbs/hr combined solids flow rate and 21.9 lbs/hr water. The reactor jacket was maintained at 100° C. and the residence time was 27 minutes. The peak temperature in the reactor was 67° C.

The material was then continuously flowed into a vibrating fluid bed dryer with an inlet air temperature of 82° C. The resulting product had a manganese content of 24.0 wt % and a total moisture of 0.7 wt % water corresponding to the non-hydrated manganese glycinate complex.

### Example 3

### Copper Glycinate Complex

Copper sulfate pentahydrate, glycine, and water were continuously fed to a Hydramix IR-6-6 with the following flowrates: 196 kg/hr combined dry ingredient flow and 2.0 kg/hr water. No external heat was provided and the residence time was 14 minutes. The peak temperature in the reactor was 22° C. The material was then placed in trays and set in a dryer at 50° C. and allowed to dry overnight. The resulting free flowing, granular product had a copper content of 25.5 wt % and a total moisture of 0.6 wt % water. The metal content corresponds to the dihydrate of the copper glycinate complex.

Moisture gain and hygroscopicity after manufacture are undesirable as this could change the product composition, cause material handling problems and lead to reduced shelf-life (caking issues). Hygroscopicity was evaluated by performing moisture gain and caking tests under room temperature (RT: 25° C., 60% relative humidity) and accelerated conditions (AT: 45° C., 75% relative humidity) from one to seven days. By comparing a copper glycinate complex produced as described in above to commercially available copper glycinates, it was confirmed that the process disclosed herein produces a copper glycinate hydrate that is different than the commercially available complexes.

Table 3, for example, is a summary of the room temperature results up to seven days. Competitor Product 10 and Competitor Product 11 gained notable moisture at room temperature while Product 12, prepared by the process disclosed herein, did not.

TABLE 3
Moisture Gain and Caking Results from Room Temperature
(RT: 25° C., 60% relative humidity) Tests.

| | Avg Weight Gain (wt %) | | |
|---|---|---|---|
| Sample Description | 1 day | 3 day | 7 day |
| Competitor Product 10 | 3.0 | 3.8 | 4.1 |
| Competitor Product 11 | 3.3 | 3.6 | 3.9 |
| Product 12 | 0.2 | 0.1 | 0.2 |

* Avg. Weight Gain (wt %) is calculated using day 0 as baseline.
** Gain CV (7 day; wt %) was 32 for Product 10, 3.8 for Product 11, and 12 for Product 12. CV = Coefficient of Variance = Standard Deviation ÷ Average

Table 4 is a summary of the accelerated humidity (AT) results for samples tested. Like at RT, Competitor Product 10 and Competitor Product 11 gained significant moisture while Product 12 did not. All formed soft cakes at AT. Hygroscopicity results from Product 12 are the best to-date and suggest a dihydrate (12.7% moisture) will result in minimal moisture gain/caking. These results suggest Competitor Product 10 and Competitor Product 11 are not producing moisture stable forms leading to significant moisture gain and caking.

TABLE 4
Moisture Gain and Caking Results from Accelerated
(AT: 45° C., 75% relative humidity) Tests.

| | | | | Pre-Test | Pre-Test |
|---|---|---|---|---|---|
| | | | | Moisture | Moisture + 7 |
| Sample | Avg. Weight Gain (wt %) | | | Estimate | day Gain |
| Description | 1 day | 3 day | 7 day | wt % | wt % |
| Competitor | 5.4 | 5.6 | 7.1 | 12.3 | 19.4 |
| Product 10 | | | | | |
| Competitor | 6.1 | 6.4 | 6.5 | 8.5 | 15.0 |
| Product 11 | | | | | |
| Product 12 | 0.1 | −0.1 | 0.0 | 12.7 | 12.8 |

* Avg. Weight Gain (wt %) is calculated using day 0 as baseline.
** Gain CV (7 day; wt %) was 2 for Product 11, 1.6 for Product 12, and 13 for Product 13. CV = Coefficient of Variance = Standard Deviation ÷ Average

Thermogravimetric analysis (TGA) and loss on drying (LOD) have been used to try to quantify the amount of water of hydration and to evaluate hydrate/product degradation. Product moisture stability (hygroscopicity) was evaluated by performing moisture gain and caking tests under room temperature (RT: 25° C., 60% relative humidity) and accelerated conditions (AT: 45° C., 75% relative humidity) from one to seven days. TGA/LOD estimates indicate copper glycinate prototypes have two moles of water per mole of product (between 12.3-13.4% moisture). Samples have been found to be moisture stable (gain less than 1%) under RT and AT conditions. It was found that significant weight loss—thought to be the dihydrate—starts at 130° C. with full loss of the dihydrate by 150° C.

### Example 4

### Iron Glycinate Complex

Iron sulfate heptahydrate, glycine, and water were continuously fed to a Hydramix IR-6-6 with the following flowrates: 30 kg/hr combined dry ingredient flow and 2.0 kg/hr water. The external jacket was maintained at 50° C. and the residence time was 120 minutes. The peak temperature in the reactor was 42° C.

The material was then placed in trays and set in a dryer at 50° C. and allowed to dry overnight. The resulting free flowing, granular product had an iron content of 22.8 wt % and a total moisture of 1.6 wt % water. The metal content roughly corresponds to the trihydrate of the iron glycinate complex.

What is claimed is:

1. A metal glycinate complex, the metal glycinate complex comprising:
a) a metal cation;
b) an anion derived from an organic or inorganic acid;
c) glycine, wherein the glycine is coordinated with the metal cation; and
wherein the metal glycinate complex increases in weight by less than about 3% following humidity exposure.

2. The metal glycinate complex of claim 1, wherein the metal cation is selected from the group consisting of calcium, magnesium, zinc, iron, copper, manganese, sodium, potassium, cobalt, and nickel.

3. The metal glycinate complex of claim 1, wherein the metal cation is selected from the group consisting of zinc, iron, copper, and manganese.

4. The metal glycinate complex of claim 1, wherein the anion is derived from an organic or inorganic acid selected from the group consisting of formic acid, acetic acid, fumaric acid, malic acid, hydrochloric acid, phosphoric acid, and sulfuric acid.

5. The metal glycinate complex of claim 1, wherein the anion is derived from sulfuric acid.

6. The metal glycinate complex of claim 1, wherein the anion is sulfate (SO₄⁻²).

7. The metal glycinate complex of claim 1, wherein the metal cation is selected from the group consisting of zinc, iron, and copper; and the complex further comprises water coordinated with the metal cation.

8. The metal glycinate complex of claim 7, wherein there are about 1 to about 5 moles of water per mole of metal glycinate complex.

9. The metal glycinate complex of claim 7, wherein there are 1 to 3 moles of water per mole of metal glycinate complex.

10. The metal glycinate complex of claim 1, wherein the metal glycinate complex increases in weight by less than 2% following exposure to 60% relative humidity at 25° C. for about 7 days.

11. The metal glycinate complex of claim 1, wherein the metal glycinate complex increases in weight by less than 3% following exposure to 75% relative humidity at 45° C. for about 7 days.

12. The metal glycinate complex of claim 1, wherein the metal glycinate complex is selected from the group consisting of zinc glycinate trihydrate [Zn(NH₂CH₂COOH)(SO₄).3H₂O], zinc glycinate pentahydrate [Zn(NH₂CH₂COOH)(SO₄).5H₂O], manganese glycinate [Mn(NH₂CH₂COOH)(SO₄)], copper glycinate dihydrate [Cu(NH₂CH₂COOH)(SO₄).2H₂O], ferrous glycinate trihydrate [Fe(NH₂CH₂COOH)(SO₄).3H₂O], and ferrous glycinate pentahydrate [Fe(NH₂CH₂COOH)(SO₄).5H₂O].

13. The metal glycinate complex of claim 1, wherein the metal glycinate complex is zinc glycinate trihydrate [Zn(NH₂CH₂COOH)(SO₄).3H₂O] or zinc glycinate pentahydrate [Zn(NH₂CH₂COOH)(SO₄).5H₂O] and the zinc glycinate trihydrate [Zn(NH₂CH₂COOH)(SO₄).3H₂O] or zinc glycinate pentahydrate [Zn(NH₂CH₂COOH)(SO₄).5H₂O] increases in weight by less than 3% following exposure to 60% relative humidity at 25° C. for about 7 days and/or increases in weight by less than 5% following exposure to 75% relative humidity at 45° C. for about 7 days.

14. The metal glycinate complex of claim 1, wherein the metal glycinate complex is manganese glycinate [Mn(NH₂CH₂COOH)(SO₄)] and the manganese glycinate [Mn(NH₂CH₂COOH)(SO₄)] increases in weight by less than 1% following exposure to 60% relative humidity at 25° C. for about 7 days and/or increases in weight by less than 1% following exposure to 75% relative humidity at 45° C. for about 7 days.

15. The metal glycinate complex of claim 1, wherein the metal glycinate complex is copper glycinate dihydrate [Cu(NH₂CH₂COOH)(SO₄).2H₂O] and the copper glycinate dihydrate [Cu(NH₂CH₂COOH)(SO₄).2H₂O] increases in weight by less than 3% following exposure to 60% relative humidity at 25° C. for about 7 days and/or increases in weight by less than 5% following exposure to 75% relative humidity at 45° C. for about 7 days.

16. The metal glycinate complex of claim 1, wherein the metal glycinate complex is ferrous glycinate trihydrate [Fe(NH₂CH₂COOH)(SO₄).3H₂O] or ferrous glycinate pentahydrate [Fe(NH₂CH₂COOH)(SO₄).5H₂O] and the ferrous glycinate trihydrate [Fe(NH₂CH₂COOH)(SO₄).3H₂O] or ferrous glycinate pentahydrate [Fe(NH₂CH₂COOH)(SO₄).5H₂O] increases in weight by less than 1.5% following exposure to 60% relative humidity at 25° C. for about 7 days and/or increases in weight by less than 2.5% following exposure to 75% relative humidity at 45° C. for about 7 days.

17. A feed composition, the feed composition comprising a metal glycinate complex of claim 1.

18. A process of making a metal glycinate complex, the process comprising the steps of:
a) mixing the following components: (i) a metal cation and an anion derived from an organic or inorganic acid, (ii) glycine and (iii) a limited amount of water, to generate a metal glycinate complex;
b) heating, while mixing in step (a), to not more than 80° C.; and
c) drying the metal glycinate complex to remove residual water;
wherein the product comprising metal glycinate complex increases in weight by less than 3% following humidity exposure at room temperature for seven days.

19. The process of claim 18, wherein the metal cation is selected from the group consisting of calcium, magnesium, zinc, iron, copper, manganese, sodium, potassium, cobalt, and nickel.

20. The process of claim 18, wherein the metal cation is selected from the group consisting of zinc, iron, copper, and manganese.

21. The process of claim 18, wherein the anion is derived from an organic or inorganic acid selected from the group consisting of formic acid, acetic acid, fumaric acid, malic acid, hydrochloric acid, phosphoric acid, and sulfuric acid.

22. The process of claim 18, wherein the anion is derived from sulfuric acid.

23. The process of claim 18, wherein the anion is sulfate (SO₄⁻²).

24. The process of claim 18, wherein the metal cation and anion derived from an organic or inorganic acid further comprise water coordinated with the metal cation.

25. The process of claim 24, wherein there are about 1 to about 10 moles of water per mole of metal cation.

26. The process of claim 24, wherein there are 1 to 7 moles of water per mole of metal cation.

27. The process of claim 26, wherein the metal cation and anion derived from an organic or inorganic acid is selected from the group consisting of zinc sulfate monohydrate [ZnSO₄.H₂O], manganese sulfate monohydrate [MnSO₄.H₂O], copper sulfate pentahydrate [CuSO₄.5H₂O], and ferrous sulfate heptahydrate [FeSO₄.7H₂O].

28. The process of claim 18, wherein the metal cation and glycine are mixed in a 1:1 molar ratio.

29. The process of claim 18, wherein the limited amount of water is about 0.1 wt % to about 20 wt % water relative to the metal cation and anion derived from an organic or inorganic acid and glycine.

30. The process of claim 18, wherein no external heat is added during mixing.

31. The process of claim 18, wherein external heat is added during mixing.

32. The process of claim 18, wherein the mixing occurs for at least about 30 minutes.

33. The process of claim 18, wherein the metal glycinate complex is selected from the group consisting of zinc glycinate trihydrate [Zn(NH₂CH₂COOH)(SO₄).3H₂O], zinc glycinate pentahydrate [Zn(NH₂CH₂COOH)(SO₄).5H₂O], manganese glycinate [Mn(NH₂CH₂COOH)(SO₄)], copper glycinate dihydrate [Cu(NH₂CH₂COOH)(SO₄).2H₂O], ferrous glycinate trihydrate [Fe(NH₂CH₂COOH)(SO₄).3H₂O], and ferrous glycinate pentahydrate [Fe(NH₂CH₂COOH)(SO₄).5H₂O].

34. The process of claim 18, wherein the temperature of step (b) is less than about 50° C.

35. The process of claim 18, wherein the temperature of step (b) is less than about 65° C.

36. The process of claim 34, wherein the metal is zinc.

37. The process of claim 34, wherein the metal is copper.

38. The process of claim 35, wherein the metal is iron.

39. The process of claim 18, wherein the metal is manganese.

40. The process of claim 36, wherein the drying temperature does not exceed 50° C.

41. The process of claim 37, wherein the drying temperature does not exceed 110° C.

42. The process of claim 38, wherein the drying temperature does not exceed 70° C.

43. The process of claim 39, wherein the drying temperature does not exceed 200° C.

44. The process of claim 18, wherein the drying temperature does not exceed 200° C.
